(19) United States
(12) Patent Application Publication
(10) Pub. No.: US 2011/0077515 A1
(43) Pub. Date: Mar. 31, 2011

(54) TISSUE STRAIN ANALYSIS

(75) Inventors: Olivier Gerard, Viroflay (FR); Thomas Gauthiera, Seattle, WA (US); Cecile Dufour, Paris (FR)

(73) Assignee: Koninklijke Philips Electronics N.V.

(21) Appl. No.: 12/993,591

(22) PCT Filed: May 20, 2009
(86) PCT No.: PCT/IB09/52103

§ 371 Date: Nov. 19, 2010

Related U.S. Application Data
(60) Provisional application No. 61/056,945, filed on May 29, 2008.

(51) Int. Cl. A61B 8/00

(52) U.S. Cl. 600/440

(57) ABSTRACT

Elasticity variation within biological tissue is often correlated with its pathology. Variation of elasticity can be assessed by specific ultrasound acquisition sequences during which pressure is applied. The tissue motion and deformation during those sequences is correlated to the tissue stiffness. The present invention describes a hybrid method that combines a real-time monitoring mode during acquisition and a non-real-time fine analysis after acquisition. This method allows early identification and fair assessment of pathology from elastographic analysis to obtain the best possible result for elastography assessment.

### FIELD OF THE INVENTION

The present invention relates to a method of obtaining tissue strain data. This strain data can be obtained by use of an ultrasonic probe also known as a transducer. The invention also relates to a corresponding computer program product and measurement apparatus.

### BACKGROUND OF THE INVENTION

Elasticity variation within biological tissue is often correlated with its pathology. Variation of elasticity can be assessed by specific ultrasound acquisition sequences during which pressure is applied. The tissue motion and deformation during those sequences is correlated to the tissue stiffness. Breast cancer tumours for instance present more stiffness against surrounding tissue.

Elastographic analysis relates to elasticity measurement as biological tissues are submitted to an external constraint; the compression from an ultrasound probe for instance. This compression is created by the operator and thus it is virtually impossible to control the speed and extent of the exerted force. The compression is thus subject to the experience of the user in order to create a continuous constant pressure with the right intensity and speed.

The usual way to compute strain and displacement from ultrasonic images is based on Doppler effect and is making use of tissue Doppler imaging (TDI) acquisition modes. It has been shown that strain and displacement can be computed from this 1D data (in the direction of the ultrasonic signal). It has to be noted that the quality of such a strain image strongly depends on the operating conditions during the acquisition phase (e.g. operator must have the right speed and force for exerting the compression). In any case, despite optimal operating conditions, the final quality of the strain image is often less than satisfactory today.

More recent algorithms working on grey level loops (B mode data) have been proposed to compute strain in 2D. In the present description 2D can be understood to cover also 3D depending on the properties of the probe used. High quality is achieved as soon as said operating conditions are fine (e.g. right speed, etc.). The computations are however too slow to be able to run in real-time and thus they need to be performed off-line.

Accordingly, despite the 2D algorithms have the capacity to achieve good performance, the results cannot be easily obtained. In particular, the operator is able to check if the operating conditions were fine and whether new acquisitions need to be done, only when he gets the 2D strain image offline, which is well later than the acquisition phase. This thereby limits interoperability.

### SUMMARY OF THE INVENTION

According to a first aspect of the invention there is provided a method of providing strain images in an ultrasonic diagnosis system, the method comprising the following steps:
in a first operation mode of the system, carrying out ultrasonic tissue data acquisition for obtaining first type of data suitable for displaying to an operator a first type of strain image at a first display speed that is real time,
displaying to an operator the strain image of the first type at the first display speed,
once the strain image of the first type that is displayed is determined to satisfy a predetermined condition, switching to a second operation mode of the system for acquiring second type of ultrasonic tissue data suitable for displaying to the operator a second type of strain image at a second display speed which is lower than the first display speed,
displaying to the operator the second strain image at the second display speed.

Thus, the present invention describes a hybrid method that combines a real-time monitoring mode for rapidly obtaining strain images of medium quality and slower imaging mode for obtaining strain images of high quality.

As will become clear in the following, this dual-mode method allows in an embodiment an early identification and fair assessment of pathology from elastographic analysis for obtaining the best possible result for elastography assessment of a tissue. The invention thereby provides a method capable of quickly providing high quality tissue strain images.

According to a second aspect of the invention there is provided a computer program product comprising instructions for implementing the method according the invention when loaded and run on a computer or a processor of an ultrasonic system.

According to a third aspect of the invention there is provided an ultrasonic diagnostic system for obtaining a high quality tissue strain image, the system comprising:
a switch for switching the system between a first and second operation mode,

a probe for acquiring tissue data acquisition,

a display for displaying strain images and,
a processor for executing the computer program product according to the second aspect of the invention.

Other aspects of the invention are recited in the dependent claims attached hereto.

### BRIEF DESCRIPTION OF THE DRAWINGS

Other features and advantages of the invention will become apparent from the following description of non-limiting exemplary embodiments, with reference to the appended drawings, in which:

FIG. 1 shows a measurement arrangement for performing the data acquisition in accordance with the present invention;

FIG. 2 is a flow chart describing an embodiment of a method in accordance with the present invention; and

FIG. 3 shows a simplified block diagram of the ultrasonic probe in accordance with an embodiment of the present invention.

### DETAILED DESCRIPTION OF THE INVENTION

In the following description a non-limiting exemplary embodiment of the invention will be described in more detail. In this embodiment the invention is applied in breast elastography where the obtained elastogram can help to discriminate between benign and malignant tumours. It is to be noted that the invention is not limited to this application, but the invention can also be applied to echocardiography, where real-time inspection of TDI information would assess the value of acquisition and offline speckle tracking methods will further bring fine computations of relevant clinical parameters.

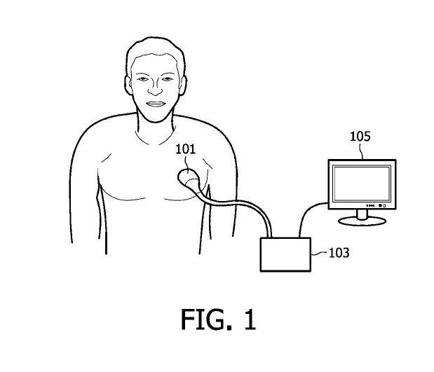

FIG. 1 shows an ultrasonic sensor probe, also known as a transducer 101 that is placed on the chest of a patient for obtaining ultrasonic signals from tissues of the patient. An ultrasonic transducer is a device that converts energy into ultrasonic or sound waves above the normal range of human hearing. The transducer 101 is connected to a processing unit 103 which is further connected to a display 105 for showing the measurement results to the operator of the transducer 101. Thus, the tissue can be compressed by the ultrasonic probe 101 handled by the operator. Real-time ultrasonic data is acquired during compression phase and displayed to the user for him to control the force exerted. The probe 101 includes a plurality of transducer elements (not shown in FIG. 1) and it may also contain a beamformer. The beamformer may also be located in the processing unit 103, which further contains echo and flow processors, a filter, an image processor and an image buffer.

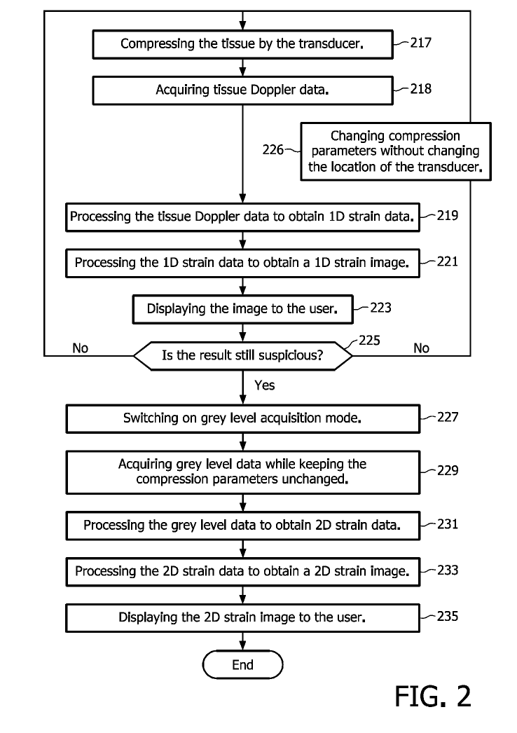

An embodiment of the invention will be next described in more detail with reference to FIG. 1 and the flow chart of FIG. 2. In this example the operator is looking for a suspicious tissue. First in step 217 the operator places the probe 101 on the patient's tissue that he believes is suspicious so that the probe is in contact with the tissue and then compresses the tissue by the probe 101. The operator may need to compress several times to obtain a desired result. Indeed, as known in the art, for obtaining tissue strained images of good quality, the operator has to manipulate in an optimal way. In particular, the probe has to compress and exert a certain force onto the skin of the body. In the invention, the wording “operating condition” will refer to these conditions of probe manipulation.

In step 218, tissue Doppler data is acquired according to an operating condition. Then in step 219 the tissue Doppler data is processed for obtaining 1D strain data. This data is further processed in step 221 for obtaining a 1D strain image, which is displayed to the operator in step 223. The displayed data can be strain and/or strain rate (in the direction of the ultrasonic probe) in the form of an elastogram. Now the operator can determine in step 225 based on the 1D strain image, whether the tissue he is investigating still seems suspicious. If he decides that the tissue is no longer suspicious or abnormal, then the operator may place the probe to another location and the process continues in step 217.

Alternatively, if the tissue is determined not to be suspicious then the operator can change the operating condition, notably the compression parameters without changing the location of the probe 101. For instance the applied force can be changed as well as the speed of the probe 101.

On the other hand if in step 225 is was determined based on the 1D strain image that the tissue still looks suspicious, then in step 227 the grey level acquisition mode is switched on. 2D strain images cannot be obtained from tissue Doppler data and therefore grey level data needs to be acquired at this stage of the process.

It is to be noted that the wording switch does not necessarily mean that there is a binary situation, namely either TDI or grey level loop. In embodiments of the invention, “switch” can mean that the weight is increased. In particular, during an acquisition step the weight of TDI data acquired can be increased compared to that of grey level data.

In step 229 the grey level data is acquired while keeping the compression parameters unchanged. Thus, the grey level data is acquired in the operating condition which has been recognized in the first operation mode to be of high quality. Next in step 231 the 2D grey level data is processed for obtaining 2D strain data. In step 233 the 2D strain data is processed so that a 2D strain image is obtained. This 2D strain image is then displayed to the operator in step 235.

In the embodiment explained above, steps 217, 219, 221, 223, 225 and 226 can be considered to form the first operation mode and steps 227, 229, 231, 233 and 235 form the second operation mode. In this example the first operation mode is performed online, i.e. in real-time, whereas the data processing in steps 231 and 233 in the second operation mode is performed in non-real-time, preferably offline. Steps 227 and 229 are performed in real-time.

Switching from the first to the second operation mode can be performed by the operator via a knob which, for example, is allowed to take two positions.

In the above example and as briefly explained above, it is also possible that in the first operation mode at least two types of data sets are acquired. In other words both tissue Doppler data and 2D grey level data can be acquired. These data sets can be acquired simultaneously. It is to be noted that if good quality tissue Doppler data is wanted, this requires a relatively long time period and for this reason the quality of the grey level data is only mediocre or even bad. Thus, if the acquisition time period is kept constant, there is quality trade-off between the tissue Doppler data and the grey level data.

A balance has to be kept between the time spent in acquiring tissue Doppler and grey level data. This balance can also be modified in the first operation mode, i.e. during the course of the examination. The acquisition can start in the first operation mode with more emphasis (weight) on real-time TDI analysis for scouting, i.e. finding the suspicious tissue. Once the suspicious tissue has been found, more emphasis can be given in step 227 on the acquisition of the grey level data so that higher quality strain images can be obtained. The emphasis can be given here in terms of the spatial and temporal resolution. For instance, if the spatial resolution is improved, this means that more scanning lines are used. If the temporal resolution of the grey level acquisition is improved, then the grey level data should be acquired as quickly as possible. The balance can be made available to the operator, e.g. by a movable knob, for example movable in rotation, mounted on the probe 101 or on the processing unit 103. One of the main target applications is breast imaging, which does not require very high frame rate, thus such a compromise can easily be found even with currently available echographs.

In the first operation mode the system is configured to provide a medium quality but real-time strain image display from the acquired data. Then by the request of the operator, the system switches to the second operation mode. In the second operation mode the data processing is typically done offline. This mode is also configured to provide a higher strain imaging quality but may be non real-time. In this way, the operator has the possibility to quickly and efficiently choose from the first mode a data set which he considers of a quality that is worth performing a more complex and non real-time algorithm providing very high quality (second mode).

The real-time feedback is important during acquisition but is a serious limitation in terms of the nature of the parameters as well as the precision that can be reached. In the second operation mode much complex algorithms can be used to derive more complex parameters. For instance it has been shown that speckle tracking technologies are able to track the motion and deformation of the tissue in 2D (and 3D), based on the grey level data.

Instead of using the tissue Doppler data and grey level data, it is possible to only use RF signals to obtain a high quality strain image, where RF signal is actually the acquired “raw” signal from which grey level (and possibly TDI, but then the size of the obtained data set will be huge) can be computed. It contains higher frequency information than grey level. However, the RF signal is rarely available from a commercially available echograph. Alternatively it is possible to combine RF and tissue Doppler data to obtain the most precise results. Whatever the selected algorithm, the teachings of the present invention can be applied to that method for computing and displaying important clinical parameters. The values of these parameters are certainly relevant because the quality of the acquisition had been checked and controlled and the best possible method was used to compute them.

If the grey-level data, i.e. B mode data, and the tissue Doppler data are acquired simultaneously during the process, this ensures that the operator does not necessarily need to carry out a second acquisition once he has identified the operating condition which leads to high quality. This is especially interesting when the operating condition defines a way to manipulate the probe onto the body of the patient (force, speed . . . ).

Above, one embodiment of the invention was described. The invention equally relates to a computer program product that is used to store computer program code for implementing any of the method steps as described above when loaded and run on computer means of the probe 101, the processing unit 103 and/or the display 105. The computer program may be stored/distributed on a suitable medium supplied together with or as a part of other hardware, but may also be distributed in other forms, such as via the Internet or other wired or wireless telecommunication systems.

The invention equally relates to an integrated circuit that is arranged to perform any of the method steps in accordance with the embodiments of the invention.

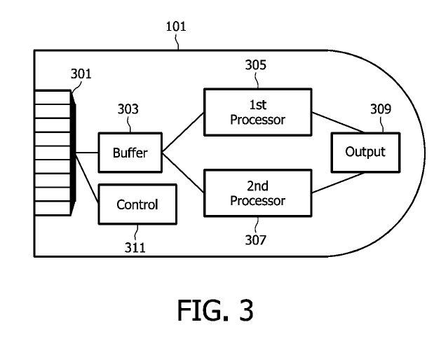

FIG. 3 is a simplified block diagram of the probe 101 showing only elements that are useful for understanding the present invention. The data acquisition means 301, i.e. the transducer elements 301 are used for acquiring the different type of data as was explained with reference to FIG. 2. The data is then buffered to a buffer 303, from where the data is fed to a first processor 305 and to a second processor 307. The first processor 305 is arranged to process data in real-time, whereas the second processor 307 is arranged to process data in non-real-time. It is also possible that a single processor is used for real-time and non-real-time processing. The processors are then connected to an output unit 309 that can transfer the processed data to the processing unit 103 for further processing and eventually for displaying the strain images. There is also shown a control unit 311 that is arranged to control the operating parameters based on the operator's input.

While the invention has been illustrated and described in detail in the drawings and foregoing description, such illustration and description are to be considered illustrative or exemplary and not restrictive; the invention is not restricted to the disclosed embodiment. Other variations to the disclosed embodiment can be understood and effected by those skilled in the art in practicing the claimed invention, from a study of the drawings, the disclosure and the appended claims.

In particular, it is recalled that a problem addressed by the invention and mentioned in the introduction is that it can be difficult for an operator to obtain a strain image of high quality. It is indeed recalled that the quality of such a strain image strongly depends on the operating conditions during the data acquisition phase (e.g. operator must have the right speed and force for exerting the compression). It is further recalled that using most recent complex algorithms working on grey level loops does not provide a satisfactory solution, notably in terms of processing time.

Thus, in order to increase the chance to rapidly get a strain image of high quality, the ultrasonic diagnostic method and system according to another embodiment of the invention may be configured as follows.

In the first operation mode, the system lets the operator carry out data acquisitions in operating conditions that he controls (e.g. speed and force for exerting the compression onto the body with the probe). In this mode the system is configured to provide from the acquired data medium quality strain images, but displayed in real-time.

Preferably, the medium quality of this first type of strain image corresponds to a quality that is obtained when performing a one dimensional strain analysis on the acquired data. For example, this first type of strain image is obtained from tissue Doppler data.

Thus, the first operation mode of the system may be seen as allowing the operator to carry out an imaging scouting process. Namely, a process in which the operator has the possibility to adjust notably the way he manipulates the probe by analyzing the first type of strain images that are displayed on the screen of the system in real-time.

Once the operator considers from a displayed strain image of the first type that he has found an optimal operating condition (for example, a manipulation of the probe at the right speed and right force against the human body), he causes the system to switch to the second operation mode. Typically, the criterion (at step 225) that the operator will use for deciding to switch in the second operation mode is related to the quality of the displayed strain image in the first operation mode. Indeed, it is recalled that if the operating conditions are non-optimal the system will display a strain image of low quality, namely the operator will have difficulty to recognize an object in the image that he would like to observe. While, as soon as the operating conditions improve, the quality of the strain image displayed by the system improves accordingly. Therefore, each time the operator adjusts the operating condition (e.g. manipulation of the probe onto the body), he can observe in real-time the impact of this adjustment on the quality of the image. It is to be noted, that in this first operation mode the strain images will necessary be limited to the said medium-quality.

Once the system is in the second operation mode, the operator causes the system to perform a new data acquisition, while he reproduces the optimal operating condition that he lastly found in the first operation mode (e.g. right speed and force for exerting the compression onto the body with the probe).

In an aspect according to the invention, in the second operation mode the system may be off-line and a complex algorithm as recited in the introduction may be used to process the new data. These new data may preferably correspond to B mode data with a high spatial resolution for displaying a second type of strain image that corresponds in this case to a B mode strain image. Thus, in this case the time needed by the system to display a strain image of a second type (display speed) is higher than the time needed in the first operation mode. However, according to the invention, by using the system in the second operation mode under the optimal operating conditions found in the first operation mode, the operator has a better chance to apply the complex algorithm on a data set which will lead to a high image quality. In other words, in the second operation mode, the operator may have to wait a certain time before the strain image is displayed, but contrary to the state of the art, he expects that quality will be achieved in one pass. In particular, in the invention the probability that the operator has to carry out a new data acquisition and run again the complex algorithm have been strongly reduced.

It is to be noted that according to the invention, the second type of data may enable to obtain a strain image based on either two or three dimensional strain processing. A two or three dimensional strain processing based image may be of better quality than a one dimensional strain processing based image.

In the claims, the word “comprising” does not exclude other elements or steps, and the indefinite article “a” or “an” does not exclude a plurality. A single processor or other unit may fulfil the functions of several items recited in the claims. The mere fact that different features are recited in mutually different dependent claims does not indicate that a combination of these features cannot be advantageously used. Any reference signs in the claims should not be construed as limiting the scope of the invention.

1. A method of providing strain images in an ultrasonic diagnosis system, the method comprising the following steps:
in a first operation mode of the system, carrying out ultrasonic tissue data acquisition for obtaining first type of data suitable for displaying to an operator a first type of strain image at a first display speed that is real time,
displaying to an operator the strain image of the first type at the first display speed,
once the strain image of the first type that is displayed is determined to satisfy a predetermined condition, switching to a second operation mode of the system,
in said second operation mode of the system, carrying out ultrasonic tissue data acquisition for obtaining second type of ultrasonic tissue data suitable for displaying to the operator a second type of strain image at a second display speed which is lower than the first display speed,
displaying to the operator the second strain image at the second display speed.

2. The method according to claim 1, wherein the first type of data enables to obtain a one dimensional processing based strain image, and wherein the second type of data enables to obtain a two or three dimensional processing based strain mage.

3. The method according to any of the preceding claims wherein, the first type of data is tissue Doppler data and the second type of data is grey level data.

4. The method according to any of claims 1 to 3, wherein the predetermined condition is whether the strain image of the first type shows a tissue suspicious to the operator.

5. The method according to any of claims 1 to 4, wherein in the first operation mode, another ultrasonic tissue data acquisition is carried out if the strain image of the first type does not shows a tissue suspicious to the operator.

6. The method according to any of claims 1 to 3, wherein the predetermined condition is whether the strain image of the first type achieves a predetermined quality.

7. The method according to any of claim 1 to 3 or 6, wherein in the first operation mode, another ultrasonic tissue data acquisition is carried out if the operator decides from the strain image of the first type that said data acquisition has not been carried out in an optimal operating condition.

8. The method according to claim 6 or 7, wherein the method further comprises, once the strain image of the first type is determined not to satisfy the predetermined condition, changing the operating condition for the data acquisition in the first operation mode, while keeping the acquisition location unchanged.

9. The method according to claim 8, wherein the operating condition corresponds to the speed and/or the force exerted by the operator with the probe onto a human body.

10. The method according to any of claims 1 to 9, wherein the second display speed is not real time.

11. A computer program product comprising instructions for implementing the following steps when loaded and run on computer means of an ultrasonic probe:
in a first operation mode of the system, carrying out ultrasonic tissue data acquisition for obtaining first type of data suitable for displaying to an operator a first type of strain image at a first display speed that is real time,
displaying to an operator the strain image of the first type at the first display speed,
after switching to a second operation mode of the system, carrying out ultrasonic tissue data acquisition for obtaining second type of ultrasonic tissue data suitable for displaying to the operator a second type of strain image at a second display speed which is lower than the first display speed,
displaying to the operator the second strain image at the second display speed.

12. An ultrasonic diagnostic system for providing strain images, comprising:
a switch for switching the system between a first and second operation mode,
a probe (110) for acquiring tissue data acquisition,
a display for displaying strain images and,
a processor for executing the computer program product according to claim 11.

13. The ultrasonic diagnostic system of claim 12, wherein the switch corresponds to a knob.

14. The ultrasonic diagnostic system according to any of claims 10 to 13, wherein the second operation mode is an off-line mode.

15. The ultrasonic diagnostic system according to any of claims 10 to 14, further comprising a movable knob for setting a balance between spatial and temporal resolution of the first and/or the second type of data.

16. The ultrasonic diagnostic system according to any of claims 10 to 15, further comprising a movable knob for putting more emphasis on tissue Doppler imaging or B mode imaging during one of the data acquisition step.
